(19) United States
(12) Patent Application Publication
(10) Pub. No.: US 2003/0050520 A1
(43) Pub. Date: Mar. 13, 2003

(54) Waste treatment process and system

(75) Inventor: Brian S. Ahern, Boxboro, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Supercritical Combustion Corporation, Woburn, MA

(21) Appl. No.: 09/950,272

(22) Filed: Sep. 10, 2001

(51) Int. Cl. C07C 4/00

(52) U.S. Cl. 585/241

(57) ABSTRACT

A system and process forms supercritical or near-supercritical mixtures of organic waste and water, and then combusts the mixture with an oxygen-containing gas at a relatively moderate pressure. Production of nitrogen oxides, carbon oxides and soot are greatly reduced compared to most conventional methods for combustion of organic wastes.

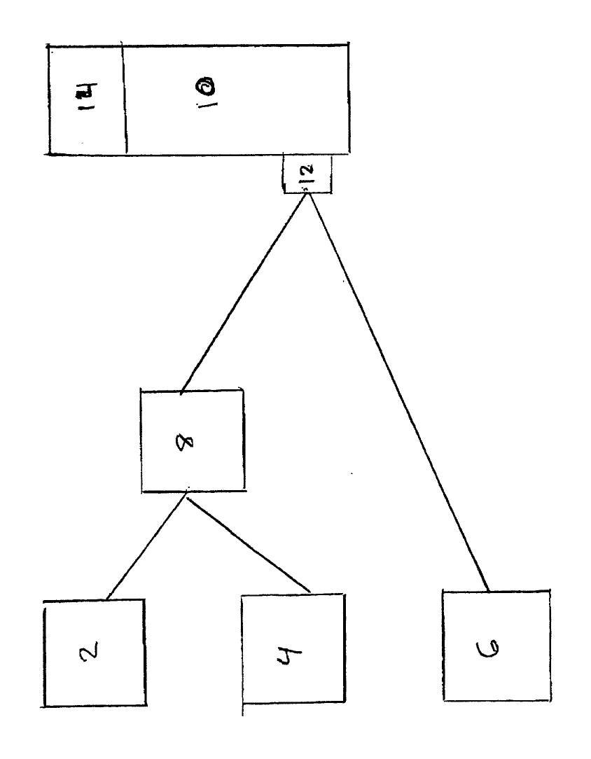

### BACKGROUND OF THE INVENTION

Hazardous and other industrial organic wastes are conventionally destroyed in large, sophisticated incinerators. Many waste incinerators are of the rotary kiln type. Both solid and liquid wastes can be introduced into the rotary kiln, in which the temperature is typically above 1800° F. Temperature is maintained at this level by using the heat content of the liquid wastes and/or by introducing supplemental fuels into the chamber, such as natural gas or diesel oil. Liquid wastes generally are pumped, generally at ambient temperature and sufficient pressure to convey the material, into the kiln through nozzles, which atomize the liquids into fine droplets. Solid wastes may be fed into the kiln in bulk or in containers, using either a conveyer or a gravity feed system. The kiln slowly rotates so that the solid wastes are tumbled, to assure that they are exposed on all sides to the high temperature in the kiln. A large fan draws excess air (containing oxygen) into the system to increase combustion efficiency. The flame and high temperature in the kiln cause the organic and some of the metal wastes to be converted from solids or liquids into hot gases. These hot gases typically are then passed into an afterburner. Any inorganic materials that have not been converted into gases drop out as ash at the end of the kiln, into a container, for further management. Atomized liquid wastes and/or supplemental fuel are typically injected (again generally at ambient temperature and sufficient pressure to convey the material) into an afterburner, where temperatures are typically maintained at 2200° F. or higher. These atomized liquids and the hot gases entering the afterburner from the kiln are mixed with air and passed through the hot flame in the afterburner. The heat and flame function to essentially break down the chemical bonds of the gaseous and atomized organic compounds into atoms. The intent is that these atoms will recombine with oxygen from the air in the chamber to form stable compounds primarily composed of non-hazardous chemicals such as carbon dioxide and water (i.e., steam). The gases exiting the secondary chamber are cooled and cleaned in an air pollution control system (APCS). The APCS is designed to remove particulates (small solid matter) and the remaining hazardous constituents—such as metals which were not destroyed by the incineration process—down to levels established as safe by the U.S. EPA/State regulations and the facility's permits.

Because of the requirement or desirability of achieving high destruction/removal efficiencies (DREs), particularly for hazardous wastes, waste combustion units include extensive and costly APCS as described above and the incineration is carried out very high flame temperatures to achieve specified DREs. The incineration facility's operating permit typically specifies the wastes that may be incinerated and establishes a DRE requirement, based on a specified principle organic constituent (POC) of one of the more difficult to destroy approved waste steam constituents. The minimum temperature of operation is typically specified in a specific requirement in a facility's operating permit. Although these high temperatures have been necessary or desirable to achieve the mandated DREs, a negative consequence is the increased generation of NOx from nitrogen bearing compounds in the waste stream and or the air used in the combustion process.

The generation of pollutants in the incineration is not only undesirable from the environmental perspective, it also can impede the recovery and beneficial use of heat generated in the process (e.g., the recovery of heat from the off-gas or combusted waste stream is impeded by the particulate/ soot commonly present).

Incineration of organic wastes containing relatively low amounts of water is economically feasible. However, with highly aqueous wastes, the energy and other costs have generally made such streams prohibitively expensive to incinerate.

Organic wastes can also be processed using supercritical liquid combustion, which involves passing oxygen or air into a dilute supercritical mixture of the wastes in water. This reaction is carried out inside very high pressure (>3200 psig) high alloy devices which are extremely expensive to build. Even at these high pressures, the organic content of the waste stream is generally must be limited to <5 weight %. The necessarily dilute nature of the system requires that a large amount of water per pound of waste be heated and cooled. Also the final temperature after combustion is low, so that the heat of combustion can be only be discarded or used only for low value applications such as space heating.

Combustion of supercritical water/fuel mixtures for energy applications is disclosed in U.S. Pat. No. 6,010,544, issued on Jan. 4, 2000 to Haldeman, et al., which is incorporated herein by reference in its entirety. Many of the advantages of supercritical water/fuel mixtures also can be obtained with sub-critical water-fuel mixtures. Many of the advantages of supercritical water/fuel mixtures also can be realized employing a mixture somewhat below the critical pressure of the mixture Ad(referred to as a sub-supercritical mixture). Such a mixture has a temperature being at least the greater of about 250° C. (482° F.) and the boiling point temperature of water at the mixture pressure. Combustion of sub-critical water/fuel mixtures are disclosed in co-pending U.S. Pat. No. 6,240,883, which is incorporated herein by reference in its entirety.

### SUMMARY OF THE INVENTION

The invention generally is related systems and processes for processing organic wastes. In one embodiment, the invention relates to systems for treating waste containing organic material, comprising one or more sources of waste-containing organic material, water and oxygen; means for forming a supercritical or sub-supercritical water/waste mixture; a waste combustion unit for combusting the mixture at a pressure below about 1000 psig; and means for injecting the water/waste mixture and oxygen into the waste combustion unit.

In another embodiment, the invention relates to processes for treating waste-containing organic material by combining waste-containing organic material and water, under sufficient heat and pressure to form a supercritical or sub-supercritical mixture thereof, injecting the mixture and oxygen into a combustion zone, and combusting said mixture at a pressure below 1000 psig.

Supercritical combustion by the method of the invention allows the combustion to be carried out in conventional incineration equipment, frequently already in place, but with drastically reduced pollution from pollutants such as nitrogen oxides (NOₓ, where x is an integer), carbon monoxide (CO) and soot. High value uses for recovered heat (e.g., steam generation for producing power), which may have been impeded by the soot, are enhanced. Use of particle removal equipment may be reduced or rendered unnecessary due to significantly reduced particulate generation form organic waste streams and the disposal of collected material lessened or eliminated.

Waste organic material may be treated by the method and apparatus of the invention in a manner that significantly reduces emissions as compared to conventional equipment and methods. Further, there is an opportunity for generating high value energy by the method and apparatus of the invention.

### BRIEF DESCRIPTION OF THE DRAWING

The FIGURE is a schematic diagram of one embodiment of supercritical combustion apparatus of the invention.

### DETAILED DESCRIPTION OF THE INVENTION

The features and other details of the invention, either as steps of the invention or as combinations of parts of the invention, will now be more particularly described with reference to the accompanying FIGURE and pointed out in the claims. It will be understood that the particular embodiments of the invention are shown by way of illustration and not as limitations of the invention. The principle features of this invention may be employed in various embodiments without departing from the scope of the invention.

Wastes containing organic-materials suitable herein include but are not limited to paint wastes, solvent wastes, process still bottoms, waste water/fuel mixtures. Some of these waste streams contain sufficient aqueous components to allow the desired water/waste ratio to be achieved. If there is insufficient water contained in a given waste, one or more sources of water can be readily used to facilitate the creation of mixture at the desired water content (e.g., as part of the means or process used for forming the mixture).

Waste combustion units suitable herein include but are not limited to rotary kiln incinerators, vertical furnaces, stokers and horizontal furnaces. The water/waste mixtures of this invention can be injected into such units in the same or similar manner as used to inject the conventional wastes (e.g., a supercritical water /waste mixture can be injected into the rotary kiln of a rotary kiln waste combustor or into an associated afterburner). Supplemental heating (e.g., by the injection of natural gas or fuel oil into the combustion zone) may also be provided, again in the same or similar manner as used in conventional waste combustion units.

Oxygen may be supplied to the combustor from any of a variety of oxygen containing gases (e.g., air or oxygen per se), again in the same or similar manner as used in conventional waste combustion units and/or by adding fuel (e.g., No#2 fuel) to the water/waste mixture.

The water/waste mixtures formed by the methods of the invention are somewhat below, at or above the critical point of the mixture. The critical pressure is the pressure required to liquefy a vapor at the critical temperature. The critical temperature is the temperature above which a vapor cannot be liquefied, regardless of pressure. The point at which the temperature and pressure have their critical value is known as the critical point. Above the critical point, there is no distinction between gas and liquid phases. Fluids beyond the critical point are known as supercritical fluids. Critical points can be obtained from the literature or determined experimentally, as known in the art. For example, phase diagrams, showing the critical point, are available in the literature (e.g., Volumetric and Phase Behavior of Hydrocarbons, Gulf Publishing Company, 1949) or can be generated experimentally for pure substances or mixtures of substances.

The critical point of water is about 374° C. (705° F.) and 3221 psi (220 atmospheres). Organic wastes generally include compounds having a wide range of molecular weights and as such do not have a well-defined critical temperature. The addition of a liquid hydrocarbon or other organic material to water results in an altering of the critical temperature and critical pressure of the water/waste mixture compared to the individual components. For example, the critical temperature of a 50/50 weight percent mixture of waste No. 2 hydrocarbon fuel and water is about 363° C. Generally, the critical temperature of a water/fuel mixture is approximately equal to the weighted average of the critical temperatures of each of the fluid species and is generally in the range between about 250° C. and 600° C., depending upon the molecular composition and percentage of hydrocarbon. However, as in the above cited 50/50 mixture (or other mixtures of two chemically dissimilar mixtures), the critical temperature of the mixture can be significantly lower than the simple weighted average due to the entropy change resulting from the chemical bonds formed between the components. Often, mixtures of one or more liquid hydrocarbons and water have a lower critical temperature than that of pure water. For example, the critical point of a mixture of 25 weight % water and 75 weight % No. 2 fuel oil is about 362° C. (684° F.) at 3300 psi. As previously noted, a 50/50 weight percent mixture of water and No. 2 fuel oil has a critical temperature of about 363° C. at 3000 psi.

In a preferred embodiment, the water/waste is at a temperature at or above about 362° C. (684° F.). Water/waste mixtures can be prepared by the method of the invention as follows. Water is separately heated to a temperature of above about 204° C. (400° F.), most preferably above about 315° C. (600° F.). In a preferred embodiment, water is heated to a temperature that does not exceed about 427° C. (800° F.). Water also can be heated above 427° C. (800° F.), for example to about 538° C. (1000° F.), and even to about 593° C. (1100° F.).

In one embodiment of the invention, the waste stream is not separately heated prior to being combined with the heated water. For example, liquid organic waste is provided at ambient temperature (i.e., the temperature will be that of the surrounding environment). In some cases (e.g., wastes stored outside in colder climates) the ambient temperature may be considerably lower than standard conditions (e.g., 70° F.) and the liquid organic waste may be heated to facilitate pumping of the waste.

The pressure of each stream is selected to ensure that the water stream and the waste stream are forced through the conduits and any associated heating means. An essentially constant pressure process is preferred, to minimize pumping difficulty and power requirements. Most preferably, the pressure is sufficiently high to facilitate the injection of the water/waste mixture into the selected combustion unit. Typically, the water is heated at a pressure between about 3200 pounds per square inch gauge (psig) and about 4000 psig. Generally, the waste stream is preferably at the same or at a similar pressure. In a preferred embodiment, the water and waste streams are each pressurized to essentially the same pressure, for example in the range of from about 3200 to about 5000 psi.

Pressures such as those employed herein can be obtained as known in the art. Suitable equipment includes, for example, compressors and pumps.

The heated water is then combined with the unheated, (i.e., ambient temperature), or heated waste. The ratio of waste to heated water used to form a water/waste mixture in the sub-critical, critical or supercritical state, can be adjusted to obtain a mixture having a selected temperature, as determined by the temperature of the individual streams forming the mixture (depending upon the temperature to which each separate stream has been heated). For example, a mixture having a temperature of about 399° C. (750° F.) and including 50 wgt. % waste No.2 fuel and 50 wgt. % water can be formed by combining 50 wgt. % waste No.2 fuel at 177° C. (350° F.) and 50 wgt. % water at about 593° C. (1100° F.), both having been previously pressurized to 4000 psi.

Means for controlling flow rates of the waste and heated water streams to form a desired water-to-fuel ratio are known in the art. Examples include flow control loops and positive displacement pumps.

The waste and water streams are combined by a suitable means to form a supercritical mixture. As defined herein, a “supercritical mixture” is a mixture of organic material and water at a temperature and pressure that exceed the critical temperature or pressure of the mixture. The critical temperature of a supercritical mixture is that temperature above which no amount of pressure will cause it to revert to the liquid state. The critical pressure of a supercritical mixture is that pressure below which a liquid state would continue to exist at a temperature up to but not above the critical temperature of the mixture. Examples of suitable means include introduction into a pipe tee, a static mixer, a dynamic mixer or by some other suitable mixing means or method known in the art for combining fluid streams.

In one embodiment of the invention, at least about 25 percent by weight of the supercritical mixture is waste-containing organic material. In another embodiment, between about 30 percent and about 70 percent by weight of the supercritical mixture is waste-containing organic material.

Optionally, the water/waste mixture is further heated. In embodiments in which the mixture is heated to a supercritical state or to a relatively high temperature sub-supercritical state, it is preferable to initially heat the water stream, prior to mixing with the waste, to about the temperature desired for the ultimate mixture and thereafter further heating the mixture to the desired final temperature. This approach is more efficient in that the heat exchange surface is minimized and the highest temperature of the heat exchanger metal is minimized (e.g., thus allowing the use of lower cost heat exchangers). Such mixtures generally have final temperatures in a range of between about 399° C. (750° F.) and about 427° C. (800° F.). When a relatively low temperature sub-supercritical mixture is being prepared, it is generally preferable to heat the water prior to mixing and not provide supplemental heating of the final mixture. Generally, the water/waste mixture can be heated without significant fouling as long as the heated surfaces containing the mixture do not exceed about 454° C. (850° F.).

Separately heating the water stream and optionally the waste stream and/or further heating the resulting water/waste mixture can be by any suitable means known in the art. In a preferred embodiment, heating of either or both of the individual waste and water streams, and/or further heating the resulting mixture is conducted by use of heat derived directly or indirectly from a combustion process employing at least a portion of the heated waste /water mixture previously formed. The means for heating and means for pressurizing of the water and/or waste streams are generally individual units, which normally would be integrally connected (e.g., by piping), however, a single unit which provides both heating and pressurizing may be used.

It is most preferred that the water stream be in the liquid or supercritical state (as opposed to the gaseous state) when heated. When the water phase is maintained as a liquid until at or near supercritical or sub-supercritical conditions, less heat needs to be put in and/or it can be put in more cost-effectively at a lower temperature.

In preferred embodiments of the invention, heat is recovered from one or several points or locations in the combustion unit and employed to heat the water stream. Optionally, or alternatively, heat transferred from the combustion unit can be employed to heat the fuel stream and/or to further heat the resulting water/fuel mixture. Several streams can be heated or further heated through well-known heat recovery/heat transfer arrangements, e.g., is recovered from the combustion exhaust.

As schematically shown in the FIGURE, a system for treating waste-containing organic material is provided, comprising one or more sources of waste containing organic material (2); one or more sources of water (4); one or more sources of oxygen (6). Means (8) for forming a supercritical mixture of the water and the waste is also provided, said means being is integrally connected through conduits not shown, to waste combustion unit (10), which may include an after-burner (14), for combusting said mixture. The mixture is injected into waste combustion unit (10) by means (12) for injecting said mixture and oxygen. Forming means (8) may included heating and/or pressurizing means, and/or heating and/or pressurizing means may be included in the waste source (2) and/or water source (4).

### EXEMPLIFICATION

Paint solvent organic waste comprising 36 weight % water and 64 weight % organics is received. The waste is placed in a 10 liter Nalgene bottle and pumped by one channel of a three-channel Eldex model BBB-4VS proportioning pump at 50 cm/min. Since the water content of the waste is sufficient to form a water/waste mixture of the suitable composition, no additional water is needed. (If additional water were desired, a water stream could be merged with the waste and mixed in a single pipe). The water/waste mixture is heated to about 400° C. by electrical bayonet heaters in a heat exchanger. The heating system is approximately isobaric from the outlet of the pump to the valve, at a pressure of about 3200 psig. These conditions are above the critical conditions of the waste/water mixture.

The mixture was fed through an injector in the form of an adjustable needle valve and a 16 mil orifice into a combustion area which consists of a 3″ diameter 24″ high quartz tube, together with a stream of air fed at about 25 SCFM. A flame is ignited with a propane torch. After removal of the torch the mixture is found to continue to combust with a flame that is clear under room light and blue in the dark. Combustion product samples are continuously withdrawn to an Enerec model 3000 emissions analyzer at a rate of about 650 cc/min. The samples are found to contain 12 ppm oxides of nitrogen (NOₓ), 1 ppm of unburned hydrocarbons and nil ppm carbon monoxide (CO) with the limit of detection of about 0.1 ppm. A three liter sample is drawn through an AVL Smoke Meter model 415 and found to indicate a filter smoke number (FSN) of zero with the limits of detection being about 0.01 FSM, in accordance with ISO draft 10054.

The waste flow is then bypassed around the heater and the water flow cut off. The resulting flame is bright yellow with flying sparks. The NOₓ is found to be 90 ppm, the unburned hydrocarbons off scale and the CO 120 ppm. The Smoke Meter reads 2.5+/−0.3.

Equivalents

While this invention has been particularly shown and described with references to preferred embodiments thereof, it will be understood by those skilled in the art that various changes in form and details may be made therein without departing from the scope of the invention encompassed by the appended claims.

What is claimed is:

1. A system for treating waste containing organic material, comprising:
(a) one or more sources of waste-containing organic material;
(b) one or more sources of oxygen;
(c) means for forming a supercritical mixture of water and said waste;
(d) a waste combustion unit for combusting said mixture at a pressure below about 1000 psig; and
(e) means for injecting said mixture and oxygen into said waste combustion unit.

2. The system of claim 1, wherein at least about 25 percent by weight of said supercritical mixture is waste-containing organic material.

3. The system of claim 1, wherein between about 30 percent and about 70 percent by weight of said supercritical mixture is waste-containing organic material.

4. The system of claim 1, wherein said means for forming a supercritical mixture includes a heating means and a pressurizing means.

5. The system of claim 1, further comprising one or more sources of water to form said supercritical mixture.

6. The system of claim 1, wherein said supercritical mixture is at a temperature of greater than about 362° C. and a pressure between about 3000 and about 4000 psig.

7. The system of claim 1, wherein said waste containing organic material further comprises an aqueous component which constitutes some or all of the water provided by said source of water.

8. The system of claim 1, wherein said waste combustion unit is an incinerator.

9. The system of claim 1, wherein said waste combustion unit further comprises at least one source of supplemental fuel.

10. The system of claim 1, wherein said waste combustion unit is an incinerator having a primary combustion chamber and an afterburner and said means for injecting said supercritical mixture into said afterburner.

11. A system for treating waste-containing organic material, comprising:
(a) one or more sources of waste-containing organic material;
(b) one or more sources of oxygen;
(c) means for forming a sub-supercritical mixture of said water and said waste, said mixture having a mixing pressure below the critical pressure of the mixture and a temperature at least the greater of about 250° C. (482° F.) and the boiling point temperature of water at the mixture pressure;
(d) a waste combustion unit for combusting said mixture at a pressure below 1000 psig; and
(e) means for injecting said mixture and oxygen into said waste combustion unit.

12. A process for treating waste containing organic material, comprising the steps of:
(a) providing waste-containing organic material and water, under sufficient heat and pressure to form a supercritical mixture thereof;
(b) injecting said supercritical mixture and oxygen into a combustion zone; and
(c) combusting said mixture at a pressure below about 1000 psig, thereby treating said waste.

13. The process of claim 12, further comprising adding supplemental fuel to the waste.

14. The process of claim 12 wherein said supercritical mixture has a temperature greater than about 362° C. and a pressure greater than about 3000 psig.

15. A process for treating waste containing organic material, comprising the steps of:
(a) providing waste-containing organic material and water to form a mixture having a mixing pressure below the critical pressure of the mixture and a temperature at least the greater of about 250° C. and the boiling point temperature of water at the mixture pressure;
(b) injecting said supercritical mixture and oxygen into a combustion zone; and
(c) combusting said mixture at a pressure below about 1000 psig.
